(19) United States
(12) Patent Application Publication
(10) Pub. No.: US 2019/0105066 A1
(43) Pub. Date: Apr. 11, 2019

(54) FOREIGN BODY RETRIEVAL DEVICE

(71) Applicant: András Kónya, Sugar Land, TX (US)

(72) Inventor: András Kónya, Sugar Land, TX (US)

(21) Appl. No.: 16/138,259

(22) Filed: Sep. 21, 2018

Related U.S. Application Data
(60) Provisional application No. 62/568,480, filed on Oct. 5, 2017.

(51) Int. Cl. A61B 17/221

(52) U.S. Cl. CPC A61B 17/221; A61B 2017/22072; A61B 2017/2212; A61B 2017/2217

(57) ABSTRACT

A method, system, and apparatus for retrieving, snaring, hooking, and/or grasping foreign bodies and retrieving them from the body of a patient. The retrieval device includes a catheter open at both ends such that one or more wires may extend at or near the distal end of the catheter and be used to retrieve the foreign body. A first, retrieval wire may be manipulated to form a loop or bend external of the catheter and around a portion of the foreign body. A second, looping wire may be manipulated to form a loop external of the catheter and around a portion of the retrieval wire, which may then be used to securely couple the foreign body and/or retrieval wire to the catheter for removal of the foreign body from the patient. The wires may extend from a side opening of the catheter and/or the end hole of the catheter.

### PRIORITY

This application claims priority to U.S. provisional patent application no. 62/568,480, filed on Oct. 5, 2017, the entire contents of which is incorporated herein by reference.

### BACKGROUND OF THE INVENTION

### Field Of The Invention

The present disclosure relates generally to the field of medical instruments for retrieving foreign articles from patients. More particularly, it concerns an apparatus for use as a snare or foreign body retrieval device that is guidewire-compatible and includes one or more continuously-adjustable loops.

### Description Of The Related Art

During medical procedures which utilize catheters, guidewires, pacemaker leads, or other medical devices, a portion of the device can sometimes break off and be left within the patient. The detached portion may then travel within the patient's vascular system, vein or artery, and comes to rest usually at a branching point or in the heart. Leaving these foreign bodies, articles, or devices (all considered “foreign bodies” for the purposes of this disclosure) within the patient can be quite harmful, and may result in complications like sepsis, perforation, thrombosis, arrhythmias, myocardial necrosis, or even death. Therefore, it is necessary and urgent to remove the foreign body from the patient. Similarly, several interventional radiological procedures involve implantation of different devices within the body. Embolization coils, stents, and vena cava filters, among others, have been frequently used. Misplacement and/or dislodgment of these devices may result in serious consequences and necessitate their removal.

The percutaneous removal of foreign bodies or devices has become acceptable because it is relatively easy and safe. However, current devices possess certain shortcomings that inhibit their ease and range of use. Several devices have been used to perform such a retrieval of foreign bodies or devices, some of which are described in U.S. Pat. No. 6,517,550 (“the '550 Patent”), incorporated herein by reference. FIG. 1 of the '550 Patent is reproduced in the present disclosure as FIG. 1A as one example of a prior art retrieval device, and FIG. 4 of the '550 Patent (showing an enlarged portion of the prior art retrieval device of FIG. 1A) is reproduced as FIG. 1B in the present disclosure. For reference purposes, FIGS. 2 and 3 from the '550 patent are reproduced as FIGS. 1C and 1D, respectively. As reproduced from the '550 Patent (and incorporated herein by reference):
“Turning first to FIG. 1, there is shown a retrieval device 10, which includes catheter 20, guidewire 30 and wire 40. A portion of wire 40 is in the form of loop 50, discussed below in greater detail. Catheter 20 has distal end 22 and proximal end 24. Catheter 20 also has lumen system 26, as illustrated in FIG. 2. As used herein, a “catheter” is any hollow tube or cover that may be placed around objects such as wires, including guidewires, and which may enclose such object(s) and prevent contact between the object(s) and the vessel or structure into which the catheter is placed. A “catheter,” as used herein, may include materials embedded within the catheter to facilitate directional control of the catheter, such as a metal braid.”
“In one embodiment, lumen system 26 may be a single lumen, as illustrated in FIG. 2. Both guidewire 30 and wire 40 may be housed and operate within the single lumen, as illustrated in FIG. 1. In another embodiment, shown in FIG. 3, lumen system 26 may include two lumens, one of which may be occupied by guidewire 30, and the other of which may house wire 40. Further, one of skill in the art will understand, with the benefit of this disclosure, that should more than two lumens prove useful for an application of the retrieval device 10, lumen system 26 of catheter 20 may be configured accordingly.”
“As shown in FIG. 1, wire 40 has distal end 42, which is hidden by catheter 20, and proximal end 44. Distal end 42 of wire 40 may be attached to catheter 20 in any suitable location using any suitable means. For example, distal end 42 may be attached to catheter 20 proximate distal end 22 of catheter 20. In this regard, distal end 42 of wire 40 may be attached to either the outside or the inside (i.e., within lumen system 26) of catheter 20 proximate distal end 22 of catheter 20. Further, distal end 42 of wire 40 may be attached to catheter 20 either closer to distal end 22 than is the hole or opening (each discussed in greater detail below) through which it is threaded, or farther from distal end 22 than is that hole or opening.”
“Distal end 42 may be attached to catheter 20 using any of a number of suitable means. For example, when catheter 20 is made from a metal (discussed below), such as nitinol, distal end 42 may be attached directly to catheter 20 by soldering, welding of any suitable style, an appropriate adhesive, or the like. When catheter 20 is made from a polymer (discussed below), such as TEFLON, NYLON, or the like, distal end 42 may be attached directly to catheter 20 by an appropriate adhesive, for example. It will be understood to those of skill in the art, with the benefit of this disclosure, that the amount of wire 40 that may be attached to catheter 20 may vary depending upon the application to which the retrieval device will likely be put.”

While conventional retrieval apparatuses (such as those disclosed in the '550 Patent) are useful in numerous instances, in certain applications they are not able to retrieve certain devices, such as when the foreign body has no free ends to grab onto and/or when each end of the foreign body is attached to a vessel wall. In particular, while the retrieval devices described in the '550 Patent offered significant advantages over other prior art retrieval devices, the '550 Patent retrieval device is only suitable for removing foreign bodies that have a free end. Referring to FIG. 1 of the '550 Patent, the loop/snare 50 is manipulated around the foreign body from its free end and grabs the free end by tightening the loop around it. The end result of foreign body retrieval from the '550 Patent is shown in FIG. 7 of the '550 Patent. Significantly, the '550 Patent retrieval device is not suitable for removing foreign bodies that do not have a free end.

There are various commercial solutions available with a loop system for the retrieval of foreign bodies. For example, the EN Snare Endovascular Snare System (offered by Merit Medical) has three fixed-diameter loops to retrieve and manipulate foreign objects in the body, while the ONE Snare system utilizes a single loop Likewise, the Atrieve vascular snare kit (offered by Argon Medical Services) provides three fixed-diameter independent loops for manipulation and retrieval of foreign bodies. These devices use nitinol, which provides flexibility, kink resistance, and torque control. Platinum strands allow for visualization under fluoroscopy. Both the Atrieve and Ensnare devices use platinum wires wrapped around a single nitinol wire Like the '550 Patent, these devices are not suitable for removing foreign bodies that do not have a free end.

The statements in this section are intended to provide background information related to the invention disclosed and claimed herein. Such information may or may not constitute prior art. It will be appreciated from the foregoing, however, that there remains a need for an improved method, device, and system for the retrieval of foreign bodies from a patient, particularly for foreign bodies having no free end and/or complicated shapes. A need exists for an improved method and system for a simply-constructed snare that is easily-maneuverable and capable of grasping foreign bodies in a reliable and minimally-invasive fashion. Such disadvantages and others inherent in the prior art are addressed by various aspects and embodiments of the subject invention.

### SUMMARY OF THE INVENTION

A method, system, and apparatus for retrieving, snaring, hooking, and/or grasping foreign bodies and retrieving them from the body of a patient. The retrieval device includes a catheter open at both ends such that one or more wires may extend at or near the distal end of the catheter and be used to retrieve the foreign body. A first, retrieval wire may be manipulated to form a loop or bend external of the catheter and around a portion of the foreign body. A second, looping wire may be manipulated to form a loop external of the catheter and around a portion of the retrieval wire, which may then be used to securely couple the foreign body and/or retrieval wire to the catheter for removal of the foreign body from the patient. The wires may extend from a side opening of the catheter and/or the end hole of the catheter.

Disclosed is a foreign body retrieval device that uses a first wire (such as a retrieval wire) and a second wire (such as a looping wire) to retrieve foreign bodies from a patient. The device may comprise a catheter having a distal end and a proximal end, a first wire positioned within said catheter and extendable through a first opening near the distal end, and a second wire positioned within said catheter and extendable through a second opening near the distal end. A distal portion of the first wire may form a predetermined shape external of the catheter after extension from the catheter, while a portion of the second wire is configured to form at least one loop external of the catheter after extension from the catheter. In one embodiment, the first and second openings may be the same, while in another embodiment the second opening is located in a side of the catheter and the first opening is an end of the catheter. The device may comprise a guidewire positioned within said catheter and extending through said distal end. The first and/or second wires may comprise a bundled wire.

Each of the first and second wires may be configured to extend and retract from the catheter. Each of the first and second wires may be configured to retrieve a foreign body from a patient. The first wire may be configured to grasp a foreign body within a patient, such as when the foreign body as positioned within the patient has no free ends. The at least one loop may be configured to couple to the distal end of the first wire. The at least one loop may have a diameter that is continuously variable based on the amount of extension of the second wire from the catheter. The at least one loop may comprise a plurality of loops, and each of the plurality of loops may be separately actuated. A distal portion of the first wire may be made of a shape memory alloy or material, such as nitinol. A distal portion of the first wire may have a preprogrammed hook shape. A distal portion of the first wire may be configured to move between a substantially linear shape and a substantially non-linear shape. A very distal end of the first wire may have a coil. A very distal end of the first wire may have a bend with an angle of between approximately 30-60 degrees. The second wire may be configured to couple the distal end of the first wire to the catheter. The second wire may have a distal end attached to the catheter at a position near the distal end of the catheter.

Disclosed is a method for removing a foreign body from a patient. The method may include advancing a catheter proximate to a foreign body in a patient, advancing a first wire (such as a retrieval wire) through the catheter to a position proximate to the foreign body, coupling the first wire to the foreign body, advancing a second wire (such as a looping wire) through the catheter to a position proximate to the first wire, coupling the second wire to a portion of the first wire, and retrieving the foreign body from the patient. More or less steps may be performed based on the particular foreign body retrieved.

The method may further comprise withdrawing the first wire and the second wire to retrieve the foreign body from the patient. The method may further comprise forming a bend of the first wire around a portion of the foreign body. The method may further comprise causing a distal end of the first wire to move from a substantially linear shape to a substantially hook shape. The method may further comprise forming a loop of the second wire around a portion of the first wire, tightening the second wire around the retrieval wire, and/or coupling a distal end of the first wire to an exterior portion of the first catheter. The method may further comprise dislodging one or more ends of the foreign body from the patient by pulling a proximal end of the first wire, and at or near the same time pulling the proximal end of the first wire. The method may further comprise forming a first loop around the foreign body by the first wire and a second loop around the first wire by the second wire. The method may further comprise inserting a guidewire into the patient, maneuvering the guidewire to an appropriate location within the patient, and advancing the first catheter over the guidewire.

Also disclosed is a foreign body retrieval device that comprises a looping wire that extends through an end of a catheter. The device may comprise a catheter having a distal end and a proximal end, wherein the catheter comprises a first opening proximate to the distal end, a cylindrical device positioned within the catheter and moveable within the catheter, and a wire positioned within said catheter having a distal end and a proximal end, wherein a proximal end of the wire is coupled to a first position of the cylindrical device and a distal end of the wire is coupled a second position of the catheter, wherein the wire is extendable through the first opening near the distal end to form at least one loop outside of the catheter. The first opening may be an end opening or a side opening of the catheter. The wire may be securely fixed to a portion of the catheter and a portion of the cylindrical device, and the wire is configured to extend outside of the catheter by relative movement of the catheter and the cylindrical device. The wire may be configured to form the at least one variable size loop by relative movement of the catheter and the cylindrical device. The first position may be proximate to a distal end of the cylindrical device, and the second position may be an inner surface of the catheter proximate to a distal end of the catheter. The wire may comprise a plurality of wires forming a plurality of loops, and may comprise a bundled wire.

Disclosed is a method for removing a foreign body from a patient that uses a continuously adjustable loop protruding from a catheter end opening. The method may comprise advancing a catheter proximate to a foreign body in a patient, advancing a retrieval wire through an opening of the catheter proximate to a distal end of the catheter, forming at least one loop in the retrieval wire outside of the catheter, advancing the at least one loop to a position proximate to the foreign body, coupling the at least one loop to the foreign body, and manipulating the at least one loop around a portion of the foreign body. The method may further comprise forming the at least one loop in the retrieval wire by relative movement of the catheter and a pushing device located within the catheter.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A illustrates one prior art retrieval device, which is taken from FIG. 1 of U.S. Pat. No. 6,517,550.

FIG. 1B illustrates an enlarged schematic of one portion of the prior art retrieval device of FIG. 1A, which is taken from FIG. 4 of U.S. Pat. No. 6,517,550.

FIG. 1C illustrates the catheter portion of the prior art retrieval device of FIG. 1A, which is taken from FIG. 2 of U.S. Pat. No. 6,517,550.

FIG. 1D illustrates a top view of the catheter portion of the prior art retrieval device of FIG. 1A, which is taken from FIG. 3 of U.S. Pat. No. 6,517,550.

FIG. 2 illustrates one schematic of a foreign body attached to a vessel wall with no free ends according to one embodiment of the present disclosure.

FIG. 3 illustrates one schematic of a retrieval device according to one embodiment of the present disclosure.

FIGS. 4A-4F illustrate using the retrieval device from FIG. 3 according to one embodiment of the present disclosure.

FIG. 5A illustrates one schematic of a retrieval wire with a bent distal end.

FIG. 5B illustrates one schematic of a retrieval wire with a twisted distal end.

FIGS. 6A and 6B illustrate top views of various embodiments of a loop or snare of the device of FIG. 3 with multiple loops.

FIGS. 7A and 7B illustrate schematics of a retrieval device according to another embodiment of the present disclosure.

FIGS. 8A-8C illustrate schematics of the retrieval device from FIGS. 7A and 7B.

FIGS. 9A-9E illustrate various schematics of wire bundles according to one embodiment of the present disclosure.

### DETAILED DESCRIPTION

Various features and advantageous details are explained more fully with reference to the nonlimiting embodiments that are illustrated in the accompanying drawings and detailed in the following description. Descriptions of well known starting materials, processing techniques, components, and equipment are omitted so as not to unnecessarily obscure the invention in detail. It should be understood, however, that the detailed description and the specific examples, while indicating embodiments of the invention, are given by way of illustration only, and not by way of limitation. Various substitutions, modifications, additions, and/or rearrangements within the spirit and/or scope of the underlying inventive concept will become apparent to those skilled in the art from this disclosure. The following detailed description does not limit the invention.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures, or characteristics may be combined in any suitable manner in one or more embodiments.

Snares or retrieval devices for retrieving foreign bodies such as fragmented catheters, guidewires or pacemaker leads, as well as stents, filters or occlusive devices such as embolization coils (all considered as “foreign bodies” for this disclosure), should be configured to be easily and safely maneuvered through a patient's vasculature or other luminal organs. Further, any loop used for grasping the foreign bodies should occupy as little space as possible within the vasculature or luminal organ so as to reduce to a minimum the potential for trauma of any kind as a result of its use. Similarly, the device used to transport the loop should also occupy as little space in the vasculature or luminal organ for the same reason. Additionally, the loop should be configured so as to be continuously adjustable and capable of grasping foreign bodies of virtually all sizes such that only one or two appropriately-sized retrieval devices will adequately address a foreign body of any possible size.

Advantageously, the presently disclosed retrieval device achieves these desirable traits and overcomes the problems with previous snares, such as a lack of guidewire compatibility coupled with an easily and reliably manipulable loop for grasping the target foreign body coupled to a separate wire, hook, or attachment mechanism for initially grabbing and/or attaching to a foreign body. Additionally, the disclosed loop(s) are continuously adjustable and, as a result, only one or two appropriately-sized retrieval devices according to the present disclosure need be kept on hand in order to safely and efficiently retrieve virtually any-sized foreign body resting within the vasculature or luminal organ of a patient, including those positioned in a way in the patient such as to have no “free ends” that can be readily ensnared by a closed loop. In that regard, the arrangement of these loops is different than conventional loop devices, such as the En Snare and Atrieve devices, which are fixed-diameter loops. The catheter, loop, and/or retrieval wire of the present retrieval device is designed to occupy as little space within the vasculature or luminal organs of the patient as possible.

The disclosed methods and apparatus allow for effective grasping of a foreign body in a patient, which may be particularly suited when the foreign body is positioned such that no free end is present to enclose by a conventional prior art closed loop retrieval device (such as that described in U.S. Pat. No. 6,517,550, incorporated herein by reference). In such a situation, an additional tool is needed for grabbing the foreign body.

In one embodiment, the present disclosure provides a retrieval wire, hook, or other similar grappling device that first attaches to the foreign body, such as by encircling a portion of the foreign body by a free distal end of the retrieval wire. After attachment and/or encircling, a loop wire may be used to close around a portion of the retrieval wire (such as near the free distal end of the retrieval wire) such as to securely couple the foreign body and/or retrieval wire to the catheter for removal of the foreign body. In one embodiment, a portion of the loop wire is advanced through a side hole within a distal portion of the catheter proximate to the tip of the catheter. In another embodiment, the loop may be advanced through the end hole of the catheter as opposed to a side hole. In one embodiment, the loop may be substantially perpendicular to a longitudinal axis of the catheter, while in other embodiments the loop may be positioned relative to the catheter at an angle less than 90 degrees, such as between approximately 30 to 60 degrees.

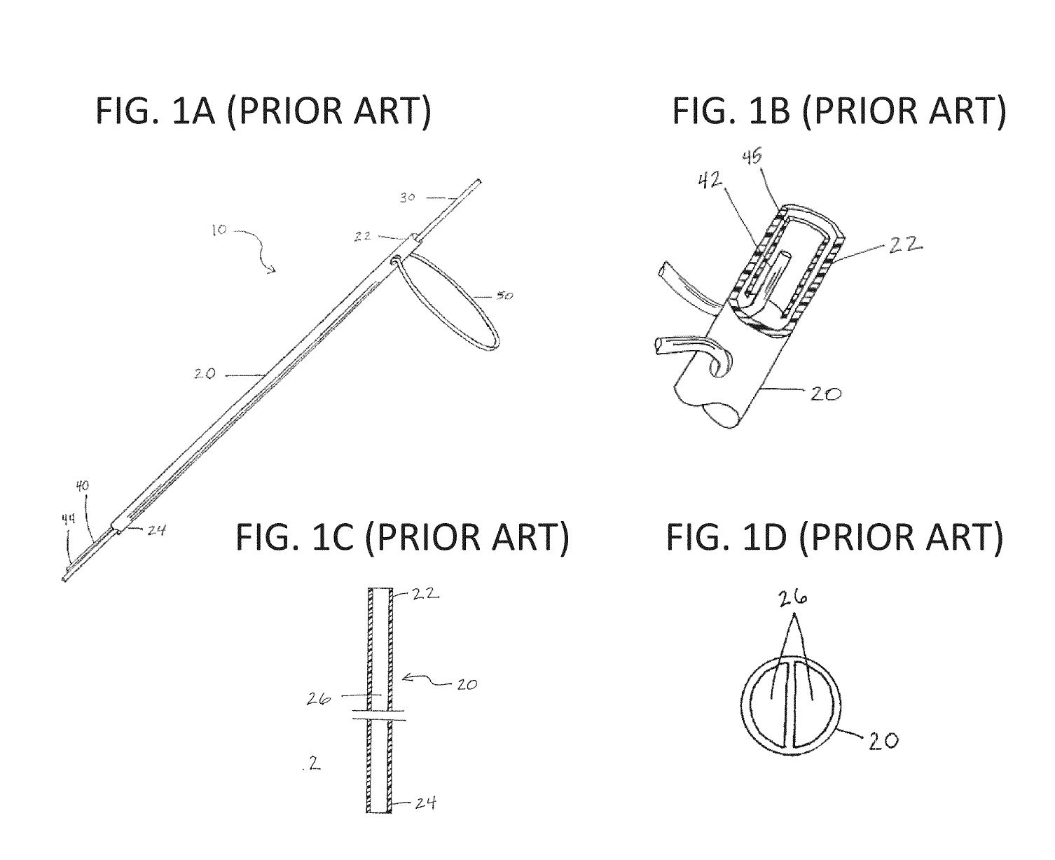

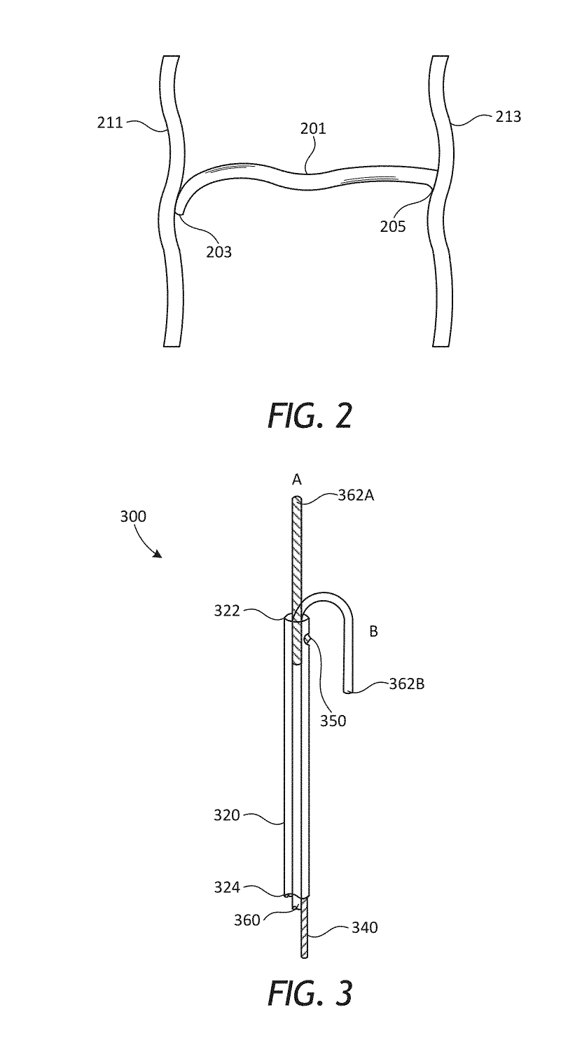

FIG. 2 illustrates one schematic of foreign body 201 within a human body or other patient (such as a pet or animal) according to one embodiment of the present disclosure. Foreign body 201 may be any number of “foreign” pieces that are not intended to be present in the patient's body, such as a broken and/or fragmented catheter, guidewire, pacemaker lead, or surgical instrument, as well as mal-deployed/migrated stents, vena cava filters or occlusive devices such as embolization coils, sheaths, and most commonly embolized central line fragments. In one embodiment, foreign body 201 may be some or all of a catheter, such as a broken catheter piece, with a plurality of ends. In one embodiment, first end 203 and second end 205 of broken catheter 201 abut and/or are attached to vessel wall 211 and 213, respectively. Other sites besides a vessel wall may include luminal organs such as stomach, small and large bowels, biliary tract, and urinary bladder. Thus, as shown in FIG. 2, foreign body 201 may have no “free ends” that serve as an end piece for a loop or snare (such as loop/snare 50 from FIG. 1A) to get around.

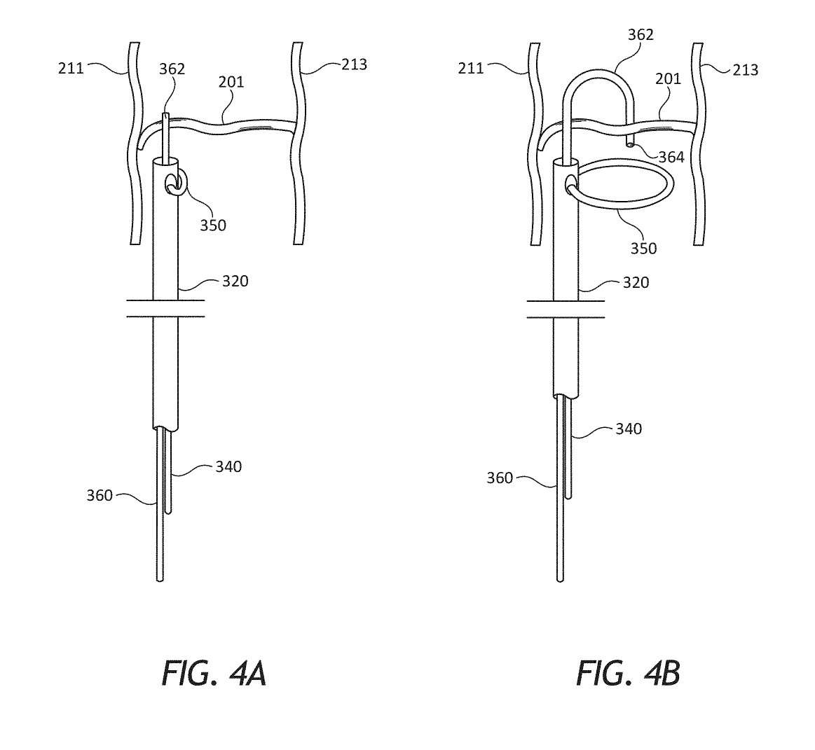

FIG. 3 illustrates one schematic of a retrieval device according to one embodiment of the present disclosure. In one embodiment, retrieval device 300 is similar to prior art retrieval device 10 disclosed in FIG. 1A, but includes an additional and specifically designed retrieval wire 360. FIG. 3 illustrates one embodiment of retrieval device 300, which includes catheter 320, retrieval wire 360, and loop wire 340. A portion of loop wire 340 is in the form of loop 350, discussed below in greater detail. Catheter 320 has distal end 322 and proximal end 324. While not shown, catheter 320 may also have a lumen system (such as lumen system 26 as illustrated in FIGS. 1C and 1D), as described in U.S. Pat. No. 6,517,550, incorporated herein by reference. As used herein, a “catheter” is any hollow tube or cover that may be placed around objects such as wires, including guidewires, and which may enclose such object(s) and prevent contact between the object(s) and the vessel or structure into which the catheter is placed. A “catheter,” as used herein, may include materials embedded within the catheter to facilitate directional control of the catheter, such as a metal braid.

Loop wire 340 has a distal end (such as distal end 42 of FIG. 1B), which is hidden by catheter 320 in FIG. 3, and a proximal end. The distal end of loop wire 340 may be attached to catheter 320 in any suitable location using any suitable means. For example, the distal end may be attached to proximate distal end 322 of catheter 320. In this regard, the distal end of loop wire 340 may be attached to either the outside or the inside of proximate distal end 322 of catheter 320. Further, the distal end of loop wire 340 may be attached to catheter 320 either closer to distal end 322 than is the hole or opening through which it is threaded, or farther from distal end 322 than is that hole or opening. The distal end may be attached to catheter 320 using any of a number of suitable means. For example, when catheter 320 is made from a metal, such as nitinol, distal end may be attached directly to catheter 320 by soldering, welding of any suitable style, an appropriate adhesive, or the like. When catheter 320 is made from a polymer, such as TEFLON, NYLON, or the like, the distal end may be attached directly to catheter 320 by an appropriate adhesive. It will be understood to those of skill in the art, with the benefit of this disclosure, that the amount of loop wire 340 that may be attached to catheter 320 may vary depending upon the intended application of the retrieval device.

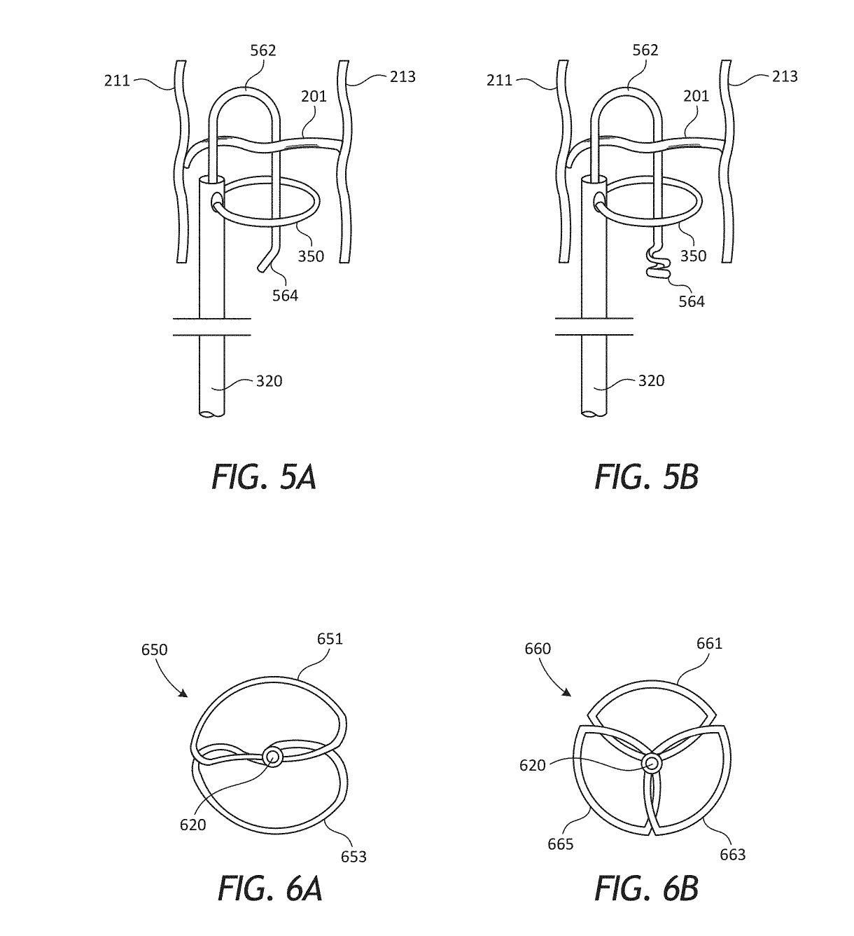

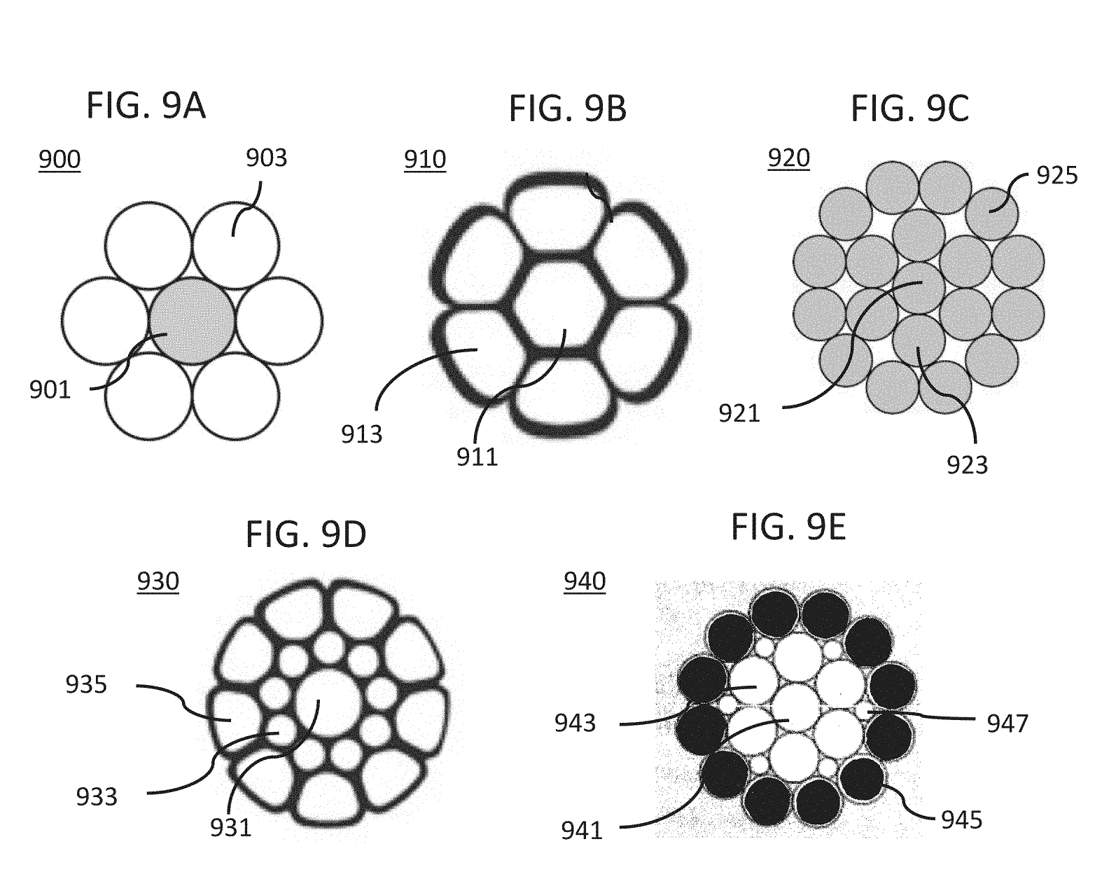

By attaching the distal end of loop wire 340 to catheter 320, loop wire 340 may be manipulated to open and close loop 350 with only one hand, as further described in U.S. Pat. No. 6,517,550. Thus, only one hand is needed to manipulate the two ends of a wire used to form a loop, and the size of such a loop is continually adjustable. Moreover, because of the continuous adjustability of the loop, loop 350 occupies as little space within the vasculature or luminal organ of the patient as possible during the manipulation and operation of the retrieval device. In one embodiment, when open or closed, loop 350 will be understood to not encircle or be wrapped around catheter 320. In some embodiments, as discussed in relation to FIGS. 6A and 6B, loop 350 may comprise a plurality of loops. Further, as discussed in relation to FIGS. 9A-9E, retrieval wire 360 and/or loop wire 340 may comprise a wire bundle.

Retrieval wire 360 is configured to grab ahold and/or otherwise attach to a foreign body before the foreign body and/or wire is ensnared by loop 350. In one embodiment, all or substantially all of retrieval wire 360 is constructed by nitinol or other shape memory material, while in other embodiments only a portion of the wire, such as a distal end of the wire, has shape memory characteristics. In one embodiment, distal end 362 of retrieval wire 360 is formed of shape memory material and/or provided with shape memory properties.

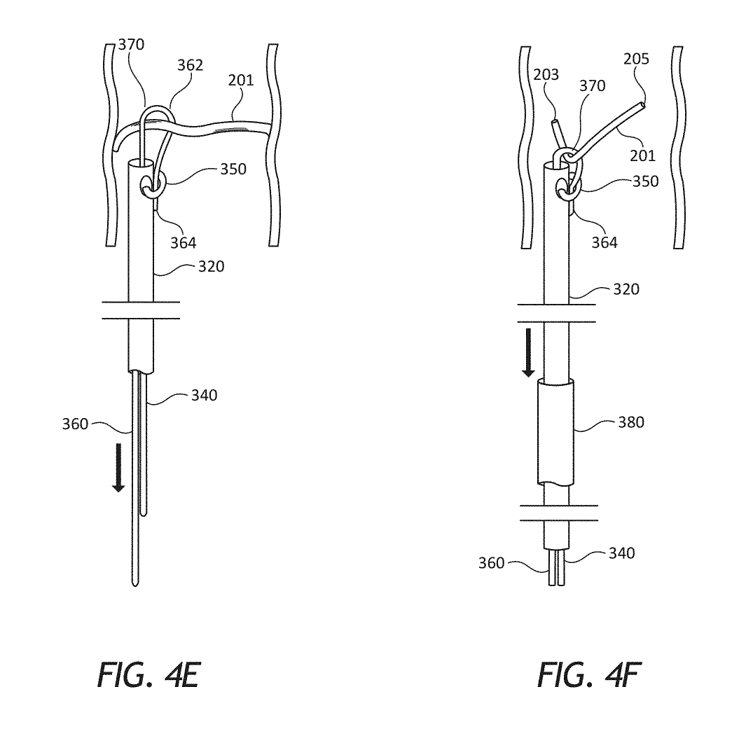

FIG. 3 illustrates two potential positions of retrieval wire 360. As retrieval wire 360 passes through catheter 320 it takes a substantially straight form. Position A of FIG. 3 shows a portion of retrieval wire 360 as it exits catheter 320 such that the shape of the retrieval wire is substantially straight and has not formed back into its preprogrammed shape. At a certain distance after the retrieval wire exits distal end 322 of catheter 320, distal end 362 of retrieval wire 360 takes its preprogrammed hook shape, as seen in position B of FIG. 3. The distal end 362 of the retrieval wire is identified as 362A in Position A and 362B in Position B. After the retrieval device is manipulated over a guidewire to the target site, the guide wire is replaced by the retrieval wire. Because the guidewire typically has an approximately 0.018-inch diameter, the retrieval wire may be made with the same size. In some situations, such as a delicate anatomy with a small caliber, the retrieval wire can be made with a slightly smaller diameter, such as between 0.012 and 0.015 inches. The working end (hook shape) of the retrieving wire may be produced with shape memory properties, and also with high radiopacity that can facilitate better visualization during manipulations guided by fluoroscopy (X-ray). For that purpose, highly radiopaque materials, platinum, tungsten, and the like can be attached to the working end of the retrieval wire, such as by using small caliber metal filament tightly wrapped around the retrieval wire. In another embodiment, the distal working profile of the retrieval wire is made of a piece of a nitinol tubing, which may be equipped with a platinum core. Appropriate heat treatment will imprint shape memory to that portion of the wire. In another possible embodiment, the very end of the working hook has a secondary bend facing toward the catheter shaft 320 after deployed the distal end 362 forming a loop shape, as shown in FIG. 5A.

While not shown in FIG. 3, the retrieval device may also have a guidewire (such as guidewire 30 shown in FIG. 1A) that is exchanged with retrieval wire 360 when the retrieval device is positioned in the vasculature (or non-vascular territory) close to the foreign body. In other words, during insertion of the retrieval device in the patient, guidewire 30 is first used within catheter 320, and as the catheter is positioned proximate to the foreign body the retrieval device 360 is switched with guidewire 30.

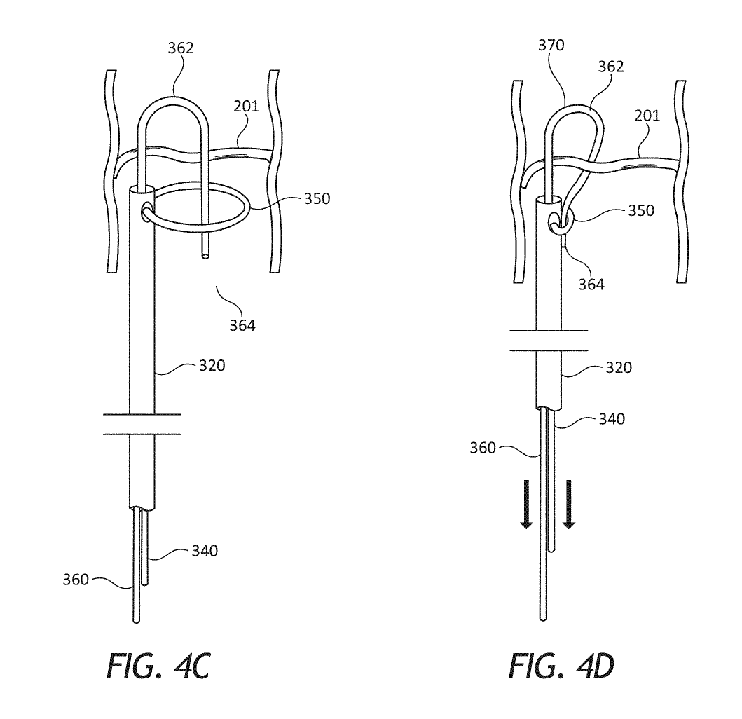

FIGS. 4A-4F illustrate using the retrieval device from FIG. 3 according to one embodiment of the present disclosure.

In one embodiment, as depicted in FIG. 4A, a retrieval device (which may be substantially similar to retrieval device 300 shown in FIG. 3) is positioned proximate to foreign body 201 with loop 350 in a closed form (e.g., the loop is substantially retracted within catheter 320 and similar to its position in FIG. 3) and retrieval wire 360 in a substantially retracted form within catheter 320. This position may be considered an initial or starting position of the retrieval device.

FIG. 4B illustrates a first position of retrieval device 300 proximate to foreign body 201 according to one embodiment of the present disclosure. Referring to FIG. 4B, retrieval wire 360 may be extended past foreign body 201, and as it extends from catheter 320 tip end 364 begins to bend back over itself into a predetermined shape. In one embodiment, loop 350 is opened out prior to retrieval wire 360 fully bending over foreign body 201. In some embodiments, loop 350 may be partially or fully opened before, after, or near the same time that retrieval wire 360 is extended around foreign body 201. Furthermore, FIG. 4B shows preformed distal end 362 bending over a portion of foreign body 201 and turning back proximally, terminating into a very distal end 364 of retrieval device 360.

FIG. 4C illustrates a second position of retrieval device 300 proximate to foreign body 201 according to one embodiment of the present disclosure. This position demonstrates after retrieval wire 360 has fully encircled a portion of foreign body 201. In this position, loop 350 has been activated just below foreign body 201 so as to encircle distal portion 362 of retrieval device 360. In this embodiment, based on the curved shape of retrieval wire 360, retrieval wire 360 enters into loop 350. Very distal end 364 of the retrieval wire may have a variety of shapes, such as shown in FIGS. 5A and 5B and described in more detail below, to facilitate grabbing the foreign body.

FIG. 4D illustrates a third position of retrieval device 300 proximate to foreign body 201 according to one embodiment of the present disclosure. In this position (after loop 350 has encircled a portion of retrieval wire 360), a proximate end of looping wire 340 is pulled back proximally to tighten snare/loop 350 maximally around a portion of retrieval device 360, such as distal end 362. As loop 350 is tightened around distal end 362, loop 350 holds, attaches, and/or otherwise couples very distal end 364 of retrieval wire 360 to an exterior portion of catheter 320. In this embodiment, loop 350 may be formed into a substantially closed position around retrieval wire 360. At this phase of the procedure, a proximate end of retrieval wire 360 is pulled proximally forming a loop of the retrieval wire (referred to as second loop 370) around foreign body 201. Thus, first closed loop 350 is formed and may be manipulated around retrieval wire 360, and second closed loop 370 is formed around foreign body 201 by retrieval wire 360. In some embodiments, both the retrieval wire 360 and loop wire 340 are pulled proximally at or near the same time for snaring the foreign body and/or retrieving the foreign body from the patient.

FIG. 4E illustrates a fourth position of retrieval device 300 proximate to foreign body 201 according to one embodiment of the present disclosure. At this phase of the procedure, a proximate end of retrieval wire 360 is pulled further proximally, thereby tightening second loop 370 around foreign body 201. In this step, loop 350 remains in a substantially closed position. In one embodiment, this closed position of loop 350 is tight enough to keep the retrieval wire attached to and/or proximate to catheter 320, but loose enough so as to allow retrieval wire to move laterally within loop 350 such that it may be tightened around foreign body 201. In one embodiment, this part of the process reduces the size of second loop 370 from a first diameter to a second, smaller diameter around foreign body 201.

FIG. 4F illustrates a fifth position of retrieval device 300 proximate to foreign body 201 according to one embodiment of the present disclosure. In this position, a proximate end of retrieval wire 360 is pulled further proximally, thereby tightening secondary loop 370 even more around foreign body 201. Secondary loop 370 is tightened to the point where foreign body 201 separates from the vessel walls, thereby separating one or more of the ends of the foreign body (such as first end 203 or second end 205) from the vessel walls. In some embodiments, the foreign body buckles and/or bends prior to breaking loose. In some instances, both ends are broken loose. At this point, the retrieval device along with the captured foreign body is removed from the patient entirely. In one embodiment, the entire foreign body capturing procedure is performed through an adequately sized thin walled sheath 380, which allows the foreign body retrieval device and the captured foreign body to pass freely through. Sheath 380 protects the foreign body from dislodging from catheter 320 during retrieval of catheter 320 from the patient. In one embodiment, sheath 380 is able to move back and forth along catheter 320 and may be configured to surround the foreign body 201 and/or distal end of catheter 320.

Other shapes and configurations of the retrieval wire are possible. For example, the end of retrieval wire 360 can be substantially straight, as shown in FIG. 3 and FIGS. 4A-4F. As another example, the retrieval wire can be formed such that the very distal segment of retrieval wire 360 turns toward the shaft of foreign body retrieval device, thereby forming a 15 to 90-degree angle, and more particularly between a 45 and 60-degree angle. This embodiment is shown in FIG. 5A, which shows a retrieval wire with distal end 562 and very distal end 564. Very distal end 564 of the retrieval wire may be substantially bent at an angle away or towards the catheter. Such a bent angle at the distal end facilitates encircling of the foreign body as well as capturing the very distal segment by loop 350. FIG. 5B shows another embodiment of the distal end of retrieval wire with a kinked and/or twisted design. In this embodiment, very distal end 564 of distal end 562 of the retrieval wire is configured into a coil (or some other non-linear/straight shape) to facilitate grabbing of foreign body 201.

Other shapes and configurations of snare/loop 350 are possible. In one embodiment, a shaft of the foreign body retrieval device (such as catheter 320) is parallel to the axis of the vascular/non-vascular conduit in which it is working, and the plane of snare/loop 350 is substantially perpendicular to the shaft. In operation, loop 350 is expanded (i.e., diameter increased) until it obtains best conformity to the diameter of the vessel.

In one embodiment, as described in relation to FIGS. 4A-4F, the foreign body removal device utilizes just one loop 350. In other embodiments, the declotting device may use a plurality of loops, such as two or three or more. Each loop may be substantially the same size in operation. In another embodiment, the loop may be formed of multiple loop segments. For example, one possible embodiment of snare 350 may consist of two or three separate loop wires that exit through side holes positioned approximately at the same circumference of catheter 320. These loops are formed in a plane that is substantially perpendicular to the axis of the device (that is to catheter 320). These loop wires are imprinted with shape memory properties that form loops 651, 653 (FIG. 6A), and loops 661, 663 and 665 (FIG. 6B).

FIGS. 6A and 6B show a top schematic view of an embodiment of the multi-loop wire with two distal loops (FIG. 6A) and three distal loops (FIG. 6B), respectively. In other words, the number of loops for the retrieval device may be one, two, or three loops, and the loops may be separate wires. In one embodiment, the use of two or more loops covers substantially all of the cross section of the vessel in which the device is utilized. Further, the use of multiple adjustable loops (similar to a single adjustable loop device as described previously herein) allows a single device to be used for a wide range of vessel sizes; thus, based on the present disclosure there is no need to produce a series of devices with different sizes. Because the loop wires for the two- and three-loop embodiments exit at the proximate end of catheter 320 laterally (in contrast to the end hole), the planes of these loops will be substantially perpendicular to the axis of the device. In that regard, the arrangement of these loops is different than conventional loop devices, such as the En Snare and Atrieve devices.

FIG. 6A shows multi-loop snare 650 with first loop 651 and second loop 653 forming two substantially equal loops at the end of retrieval wire 360 as they exit catheter 620. In one embodiment, the loops each cover approximately 180 degrees of a circle. FIG. 6B shows multi-loop snare 660 with first loop 661, second loop 663, and third loop 663, forming three loops at the end of retrieval wire 360 as they exit catheter 620. In one embodiment, the separate loops of snare 660 form substantially equal loops that each cover approximately 120 degrees of a circle. In these embodiments, the total coverage of the loops (e.g., the maximum diameter of the expanded loops together) should be selected to match as much as possible to the diameter of the conduit where the device is used to obtain maximum ensnaring coverage. The loops may be the same size or different sizes. The multi-loop embodiments illustrated in FIGS. 6A and 6B can also be used with the retrieval wire 360 the same way as described in FIGS. 4A-4F.

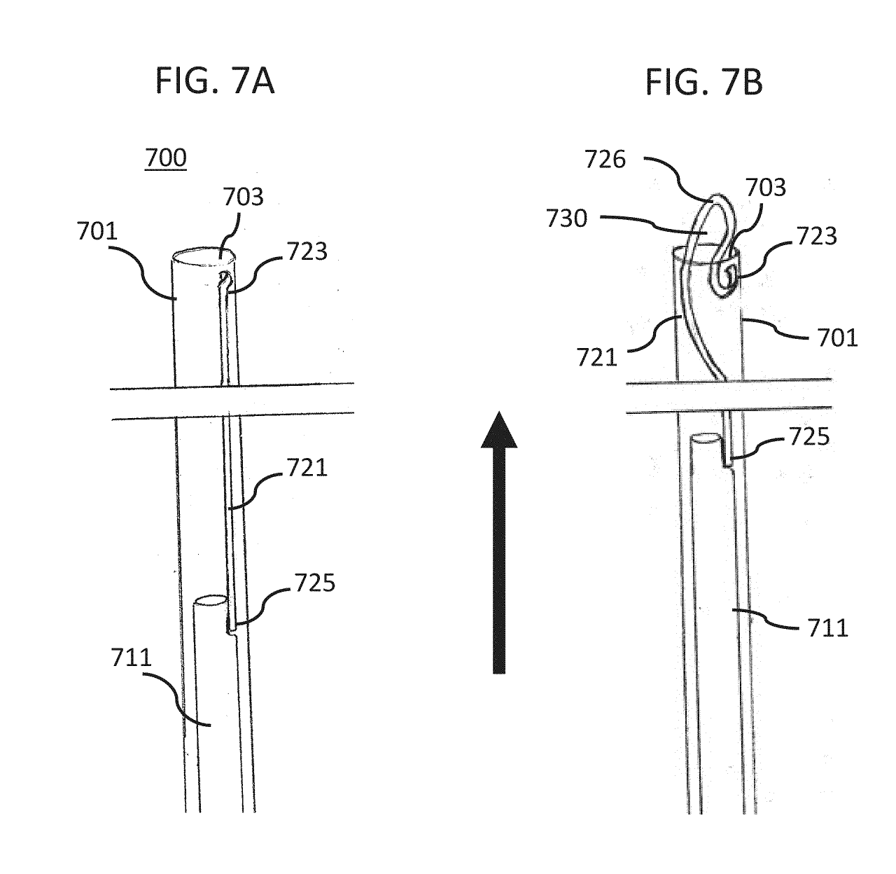

In one embodiment, the loop/snare of the device is located on a single side of the shaft/catheter, such as shown in FIG. 4B. In this embodiment, the shaft is substantially eccentrically located in relation to the loop. In other embodiments, such as shown in FIGS. 6A and 6B, the loop/snare of the device is located on multiple sides of the shaft/catheter, such that the shaft/catheter 620 may be located in a central portion of the loop and/or axis of the loop. In one embodiment, each of multi-loops 650, 660 may be activated by pushing loop wire 340 proximally out of catheter 620 until the loop is fully formed of the separate loops. In another embodiment, each separate loop (such as loop 651, 653) may be activated with a separate loop wire controlling and/or coupled to each separate loop. Such a separate control makes it possible to active only one, two, three, or some subset of the multi-loops at a given time. Multiple loops may be utilized to facilitate grabbing multiple portions of a single foreign object or even multiple objects at a single time, and in other embodiments multiple loops may be utilized to grab various portions of the same or different retrieval wires and/or foreign bodies. In some instances, the use of multiple loops allows a greater coverage area within a given diameter of the working vein or similar area to be operated within and allows greater capability to ensnare the desired foreign body object. In general, the embodiments described in FIGS. 6A and 6B may be used with the previously disclosed method and retrieval wire 360 as shown in FIGS. 4A-4F. Still further, the embodiments described in FIGS. 6A and 6B for multiple loops or loop segments may be utilized within the retrieval device embodiment illustrated below in FIGS. 7A and 7B.

In one embodiment, as described above in FIG. 3, the created loop of the disclosed retrieval device may be substantially perpendicular to a longitudinal axis of the catheter (see, e.g., FIGS. 4A-4F). In this embodiment, a mobile portion of the loop wire is advanced through a side hole within a distal portion of the catheter proximate to the tip of the catheter. In other embodiments, the looping wire may be advanced through the end hole of the catheter as opposed to a side hole, as described in more detail below.

FIGS. 7A and 7B illustrate schematics of a retrieval device according to one embodiment of the present disclosure wherein the looping wire exits an end of the catheter. FIG. 7A illustrates a schematic of retrieval device 700 in a non-advanced position. Retrieval device 700 comprises outer catheter 701 and inner catheter 711 with wire 721 coupled to each of the catheters. In some embodiments, inner catheter 711 may not be a catheter but simply some other cylindrical object and/or pushing device, such as a guide wire, super elastic wire, micro-tubing, or pushing rod, which may be solid or hollow. Wire 721 is configured to extend out end hole 703 of catheter 701 and form a loop after exit. In one embodiment, wire 721 is formed of a shape memory material, such as nitinol. During formation of the wire, it may be formed into a loop of a desired general shape. As is known in the art, in an extended or stretched position (such as within the catheter), a shape memory material may be substantially straight; whereas in a regular and/or non-extended position it may form the preprogrammed shape. A distal end of wire 721 is coupled to distal portion 723 of catheter 701 and a proximal end of wire 721 is coupled to proximal portion 725 of catheter 711. Thus, each end of loop wire 721 is secured and/or rigidly fixed to separate portions of the device. In one embodiment, the distal end of wire 721 may be attached to catheter 701 in any suitable location using any suitable means, such as being secured to an inner surface of the catheter. For example, when catheter 701 is made from a metal, such as nitinol, the distal end of wire 721 may be attached directly to catheter 701 by soldering, welding of any suitable style, an appropriate adhesive, or the like. When catheter 701 is made from a polymer, such as TEFLON, NYLON, or the like, the distal end may be attached directly to catheter 701 by an appropriate adhesive. Similar attachments may be made between a proximate portion of wire 721 and pushing device 711. Wire 721 is configured to move based on relative movement of the inner and outer catheters. By attaching the distal end of wire 721 to catheter 701, wire 721 may be manipulated in order to open and close and/or otherwise form a loop with only one hand, and such a loop is continuously adjustable in size/diameter.

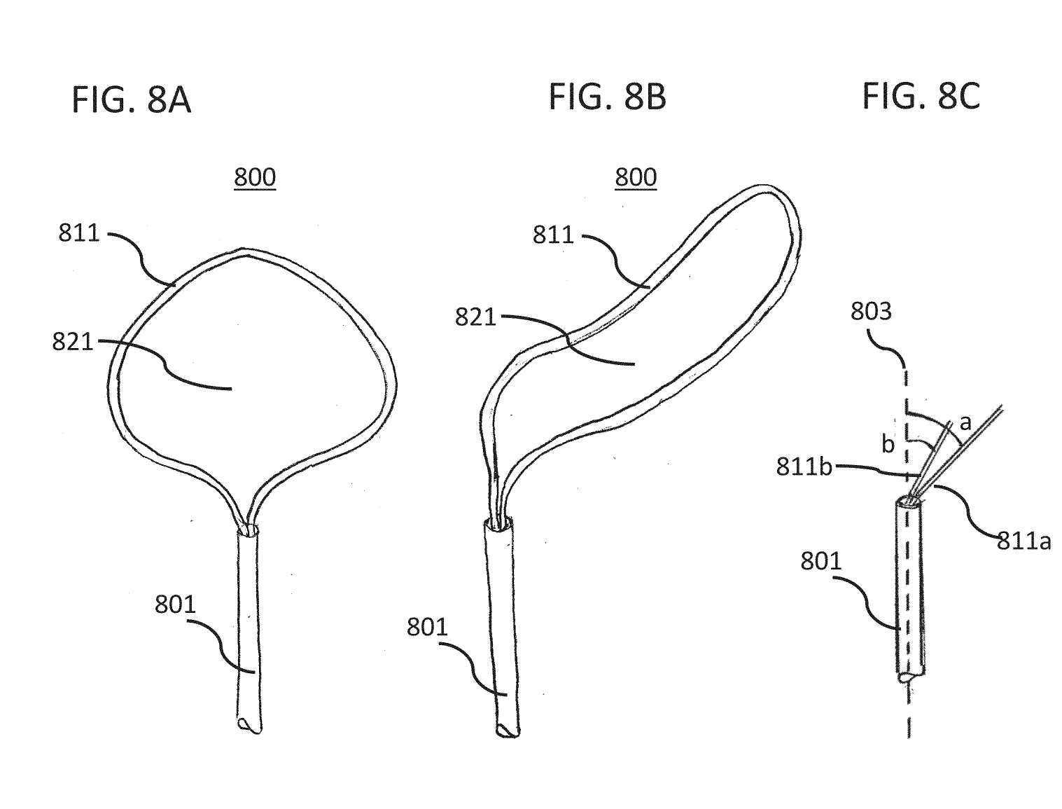

FIG. 7B illustrates the retrieval device 700 from FIG. 7A but in an extended or advanced position. In one embodiment, as pusher element/inner catheter 711 is advanced proximally (e.g., see the arrow direction in FIG. 7B), loop wire 721 is advanced and/or exits end hole 703 of catheter 701 and forms loop 730 after exit. The size of loop 730 is continually adjustable in a wide range. In one embodiment, as pushing device 711 is advanced, the size of the loop increases. In this embodiment, the created loop is not substantially perpendicular to the device/catheter (as illustrated in FIG. 3) but instead forms an angle less than 90 degrees with a longitudinal axis of the catheter, as better illustrated in FIG. 8C. Thus, FIGS. 7A and 7B illustrate two potential positions of the loop wire 721. As wire 721 passes through catheter 701 it takes a substantially straight form as shown in FIG. 7A. As shown in FIG. 7B, at a certain distance after wire 721 exits the distal end of catheter 701, the distal end of wire 721 takes its preprogrammed loop shape (or a portion thereof), as seen in FIG. 7B. Retrieval of the wire back into the catheter forces the loop again into a substantially straight form.

The retrieval device embodiment illustrated in FIGS. 7A and 7B may be used as a conventional snare/loop device. The loop may be manipulated around a free end of a foreign body and tightened until the foreign body is grabbed by and/or coupled to the loop. The foreign body and loop may be retrieved from the given anatomy by using conventional procedures.

FIGS. 8A and 8B illustrate schematics of the retrieval device from FIGS. 7A and 7B. Retrieval device 800 (which is substantially similar to device 700 illustrated in FIGS. 7A and 7B) comprises catheter 801 and wire 811, wherein wire 811 forms continuously adjustable loop 821 as it exits catheter 801. FIG. 8A shows a frontal view of retrieval device 800, while FIG. 8B shows the same loop from a lateral view. While loop 821 shows a single loop, as disclosed herein, the device may have two or three separately adjustable loops, such as those disclosed in FIGS. 6A and 6B. Both FIGS. 8A and 8B show the loop at a position where it is substantially opened. FIG. 8C illustrates a schematic of the retrieval device with a plurality of different angles of wire 811 as it exits catheter 801. In one embodiment, wire 811 may form an angle less than 90 degrees with longitudinal axis 803 of catheter 801 as it exits the catheter. In one embodiment, wire 811a extends at an angle a, while in another embodiment wire 811b may extend at an angle b (which is less than angle a). The angles may be between approximately 15-60 degrees, between approximately 30-50 degrees, or between approximately 45 to 60 degrees. In one embodiment, the device 800 can also be used with retrieval wire 360 as described earlier and illustrated in FIGS. 4A-4F.

In one embodiment, each of the looping wires and/or retrieval wires is formed of a traditional shape memory wire, such as a single wire made of nitinol. In other embodiments, the wire may be formed of a bundled wire (e.g., a set of individual wires coupled together) instead of a single wire. The bundle of wires may comprise different sized wires and/or wires of different materials. In one embodiment, rather than using a single filament or wire strand, a wire rope and/or wire bundle may be formed of a plurality of individual wire strands that are coupled together. The bundled wire may contain one or more nitinol microtubing(s) with one or more platinum wires. Each of these wire bundles may be used to form a wire for the loop as disclosed herein.

A bundle of wires instead of a single wire provides numerous benefits. For example, if one of the plurality of wires fails, the other wires can easily take up the load. Thus, any flaws in an individual wire is not as critical as compared to looking at the bundle of wires as a whole. A bundle of wires also prevents fatigue of the single individual wire, similar to the principle of rope wires (which use braided strands of individual rope filaments). As another benefit, a bundled wire provides increased tensile strength while the wire diameter can be reduced. The reduction of the wire size allows for reducing the overall dimensions/size of the device. This is important when the device is used in a small vessel, which requires smaller caliber devices. Further, for a multi-loop device, it is highly desirable to reduce the diameter of the individual wires to move them freely within the catheter and without diminishing the overall strength of the device. The reduction of the size of the loop wire can also lead more favorable physical properties. For example, a thinner diameter wire allows the same diameter loop to be made easier and earlier (e.g., using less amount of extension of the loop wire) than a larger caliber wire. As still another benefit, a bundled wire provides for increased radiopacity of the wire, which can be utilized for both the loop wire(s) and the retrieval wire. The wire bundle can contain platinum cored nitinol microtubings that significantly enhance the visibility of the devices on fluoroscopy; better visibility in turn enhances the speed, efficacy, and safety of the procedure. Further, faster removal of the foreign body translates to less radiation to the patients and the personnel. Still further, if both the loop wire and the retrieval wire are made from bundled/rope wire, the friction between them will be significantly increased, which facilitates holding the foreign body securely during retrieval based on the rough surface interaction of two wire-ropes.

There are numerous design variations of a wire bundle that can be used in the disclosed foreign body retrieval device, depending on the intended loop design, application of the retrieval device, and anatomical location in which the retrieval device is to be deployed. For example, some of the wires may be different shapes and/or sizes, while some of the wires may be different materials. Following the principle of the wire rope, virtually endless variations of a wire bundle can be created that can be used as individual wire strands for a loop of the disclosed foreign body removal device.

FIGS. 9A-9E illustrate various schematics of wire bundles according to one embodiment of the present disclosure.

FIG. 9A illustrates wire bundle 900, which may be formed of seven individual wire strands 903. Each of these strands may be twisted together helically. In one embodiment, one of the wires may comprise a nitinol microtube that may contain a platinum core used as a radiopaque marker, which is used to increase the visibility of the stent on fluoroscopy (X-ray) during deployment. For example, central wire 901 may comprise a microtube with a platinum core. In other embodiments, some or all of the wires may contain microtubings with platinum cores. The sizes of the wires 903 may be the same or different. For example, each of the seven individual wires 903 may have an approximately 0.003″ diameter (d), resulting in an overall diameter (D) of wire bundle 900 of approximately 0.009″. In another embodiment, one or more of the individual wires itself may comprise a plurality of smaller individual wires, thereby creating a more complex but stronger wire bundle (again, much like a braided rope). In one embodiment, one or more of wires 903 may comprise at least three wires, which each may comprise a diameter of 0.001″ thereby keeping the overall diameter of wire 903 at approximately 0.003″. Of course, other diameters and sizes of wires may be utilized as would be known to one of ordinary skill in the art based on the present disclosure.

FIG. 9B illustrates another schematic of a wire bundle according to one embodiment of the present disclosure. Wire bundle 910 may be substantially similar to wire bundle 900 but comprises one or more wires that are flattened, non-symmetrical, and/or non-cylindrical. For example, core wire 911 (which may or may not be compacted) may be surrounded by a plurality of flattened wires 913 that are not cylindrical, thereby providing an overall compacted shape of wire bundle 910. Among other benefits, a compacted wire bundle provides the same amount of wire with an overall less diameter than if each of the wires was not compacted. The overall amount of wire material and strength remains substantially the same between the different bundles between FIGS. 9A (non-compacted) and 9B (compacted).

FIG. 9C illustrates another schematic of a wire bundle according to one embodiment of the present disclosure. Wire bundle 920 may be substantially similar to wire bundle 900 but comprises additional wires. For example, wire bundle 920 may comprise center wire 921, a second set of wires 923 (such as six wires) surrounding the center wire, and a third set of wires 925 (such as twelve wires) surrounding the second set of wires 923. In the embodiment illustrated in FIG. 9C, there are approximately 19 individual wires, each of which may be further comprised of individual wires. Again, this embodiment is similar to wire bundle 900 but adds an outer peripheral set of wires 925.

As illustrated above, FIG. 9B illustrates a compacted wire, in which some of the individual wires are shaped to minimize the dead space within a wire bundle which exists when each of the wires are cylindrical/symmetrical. Similarly, FIG. 9D illustrates a compacted wire bundle version. Compacted wire bundle 930 comprise center wire 931, a second set of wires 933 (such as nine wires) surrounding the center wire, and a third set of wires 935 (such as nine wires) surrounding the second set of wires 933. The outer set of wires 935 comprises compacted and/or non-symmetrical wires, which reduces the overall diameter of the wire bundle 930 if all of the wires had been symmetrical. As illustrated, the second set of wires 933 has a diameter less than a diameter of the center wire and the third set of wires.

FIG. 9E illustrates another schematic of a wire bundle according to one embodiment of the present disclosure. Wire bundle 940 may be substantially similar to wire bundle 920 (FIG. 9C) but comprises additional wires. For example, like bundle 920, wire bundle 940 may comprise center wire 941, a second set of wires 943 (such as six wires) surrounding the center wire, and a third set of wires 945 (such as twelve wires) surrounding the second set of wires 943. However, bundle 940 also includes wires 947 (such as six wires) between the outer set of wires 945 and the second set of wires 943. Wires 947 may have a diameter substantially smaller than a diameter of the other wires. In one embodiment, the outer set of wires 945 microtubings with platinum cores. In other embodiments, smaller wires 947 may comprise platinum instead of the outer set of wires.

All of the methods disclosed and claimed herein can be made and executed without undue experimentation in light of the present disclosure. While the apparatus and methods of this invention have been described in terms of preferred embodiments, it will be apparent to those of skill in the art that variations may be applied to the methods and in the steps or in the sequence of steps of the method described herein without departing from the concept, spirit and scope of the invention. In addition, modifications may be made to the disclosed apparatus and components may be eliminated or substituted for the components described herein where the same or similar results would be achieved. All such similar substitutes and modifications apparent to those skilled in the art are deemed to be within the spirit, scope, and concept of the invention.

Many other variations in the system are within the scope of the invention. For example, any foreign body or device may be retrieved from a patient by use of the disclosed retrieval device, and such foreign bodies are not necessarily limited to broken catheters or other surgical instruments or foreign bodies without any “free ends.” As another example, the retrieval device may comprise a plurality of retrieval wires, such that a first retrieval wire is used on a first portion of the foreign body and a second retrieval wire is used on a second portion of the body. As another example, a single wire may form a plurality of loops or loop segments, or a plurality of wires may form a plurality of loops. The wires disclosed herein may be a single wire or a plurality of wires, such as a wire bundle. A wire bundle ay be a twisted and/or helical wire bundle. Any one or more of the wires utilized in a wire or wire bundle may have platinum or some other marker to provide increased radiopacity, such as nitinol microtubings with a platinum core. Other shapes and configurations of the retrieval wire are possible. It is emphasized that the foregoing embodiments are only examples of the very many different structural and material configurations that are possible within the scope of the present invention.

Although the invention(s) is/are described herein with reference to specific embodiments, various modifications and changes can be made without departing from the scope of the present invention(s), as presently set forth in the claims below. Accordingly, the specification and figures are to be regarded in an illustrative rather than a restrictive sense, and all such modifications are intended to be included within the scope of the present invention(s). Any benefits, advantages, or solutions to problems that are described herein with regard to specific embodiments are not intended to be construed as a critical, required, or essential feature or element of any or all the claims.

Unless stated otherwise, terms such as “first” and “second” are used to arbitrarily distinguish between the elements such terms describe. Thus, these terms are not necessarily intended to indicate temporal or other prioritization of such elements. The terms “coupled” or “operably coupled” are defined as connected, although not necessarily directly, and not necessarily mechanically. The terms “a” and “an” are defined as one or more unless stated otherwise. The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a system, device, or apparatus that “comprises,” “has,” “includes” or “contains” one or more elements possesses those one or more elements but is not limited to possessing only those one or more elements. Similarly, a method or process that “comprises,” “has,” “includes” or “contains” one or more operations possesses those one or more operations but is not limited to possessing only those one or more operations.

What is claimed is:

1. A foreign body retrieval device, comprising:
a catheter having a distal end and a proximal end;
a first wire positioned within said catheter and extendable through a first opening near the distal end; and
a second wire positioned within said catheter and extendable through a second opening near the distal end,
wherein a distal portion of the first wire forms a predetermined shape external of the catheter after extension from the catheter,
wherein a portion of the second wire is configured to form at least one loop external of the catheter after extension from the catheter.

2. The device of claim 1, wherein the first and second openings are the same.

3. The device of claim 1, wherein the second opening is located in a side of the catheter and the first opening is an end of the catheter.

4. The device of claim 1, wherein each of the first and second wires is configured to extend and retract from the catheter.

5. The device of claim 1, wherein the first wire and second wire are configured to retrieve a foreign body from a patient.

6. The device of claim 1, wherein the first wire is configured to grasp a foreign body within a patient.

7. The device of claim 6, wherein the foreign body as positioned within the patient has no free ends.

8. The device of claim 1, wherein the at least one loop is configured to couple to the distal end of the first wire.

9. The device of claim 1, wherein the second wire is configured to couple the distal end of the first wire to the catheter.

10. The device of claim 1, wherein the at least one loop has a diameter that is continuously variable based on the amount of extension of the second wire from the catheter.

11. The device of claim 1, wherein a distal portion of the first wire is made of a shape memory alloy.

12. The device of claim 1, wherein a distal portion of the first wire comprises a preprogrammed hook shape.

13. The device of claim 1, wherein a distal portion of the first wire is configured to move between a substantially linear shape and a substantially non-linear shape.

14. The device of claim 1, wherein a very distal end of the first wire comprises a coil.

15. The device of claim 1, wherein a very distal end of the first wire comprises a bend with an angle of between approximately 30-60 degrees.

16. The device of claim 1, wherein the at least one loop comprises a plurality of loops.

17. The device of claim 16, wherein each of the plurality of loops may be separately actuated.

18. The apparatus of claim 1, wherein the second wire has a distal end attached to the catheter at a position near the distal end of the catheter.

19. The device of claim 1, further comprising a guidewire positioned within said catheter and extending through said distal end.

20. The device of claim 1, wherein the first wire comprises a bundled wire.

21. The device of claim 1, wherein the second wire comprises a bundled wire.

22. The device of claim 1, wherein the first wire comprises a retrieval wire and the second wire comprises a looping wire.

23. A method for removing a foreign body from a patient, comprising
advancing a catheter proximate to a foreign body in a patient;
advancing a first wire through the catheter to a position proximate to the foreign body;
coupling the first wire to the foreign body;
advancing a second wire through the catheter to a position proximate to the first wire;
coupling the second wire to a portion of the first wire; and
retrieving the foreign body from the patient.

24. The method of claim 23, wherein the first wire comprises a retrieval wire and the second wire comprises a looping wire.

25. The method of claim 23, further comprising withdrawing the first wire and the second wire to retrieve the foreign body from the patient.

26. The method of claim 23, further comprising forming a bend of the first wire around a portion of the foreign body.

27. The method of claim 23, further comprising causing a distal end of the first wire to move from a substantially linear shape to a substantially hook shape.

28. The method of claim 23, further comprising forming a loop of the second wire around a portion of the first wire.

29. The method of claim 23, further comprising tightening the second wire around the retrieval wire.

30. The method of claim 23, further comprising coupling a distal end of the first wire to an exterior portion of the first catheter.

31. The method of claim 23, further comprising dislodging one or more ends of the foreign body from the patient by pulling a proximal end of the first wire and pulling the proximal end of the second wire at or near the same time as pulling the proximal end of the first wire.

32. The method of claim 23, further comprising forming a first loop around the foreign body by the first wire and a second loop around the first wire by the second wire.

33. The method of claim 23, further comprising
inserting a guidewire into the patient;
maneuvering the guidewire to an appropriate location within the patient; and
advancing the first catheter over the guidewire.
